(19) United States
(12) Plant Patent Application Publication
(10) Pub. No.: US 2006/0048251 P1
(43) Pub. Date: Mar. 2, 2006

(54) TORENIA PLANT NAMED 'DANTORWHITE'

(50) TORENIA HYBRID; TORENIA

(75) Inventor: Gabriel Danziger, Moshav Nir-Zvi (IL)

Correspondence Address: FOLEY AND LARDNER LLP;SUITE 500, 3000 K STREET NW, WASHINGTON, DC, 20007, US

(73) Assignee: Danziger "Dan" Flower Farm

(21) Appl. No.: 10/929,421

(22) Filed: Aug. 31, 2004

(51) Int. Cl. A01H 5/00

(52) U.S. Cl. PLT/263

(57) ABSTRACT

A new and distinct Torenia plant named ‘Dantorwhite’ particularly characterized by having shiny green foliage, dotted with plenty of delicate, cup shaped flowers in shades of white with a yellow throat; bush cascading, vigorous, compact rounded growth, densely branched; early flowering, flowers all year round in moderate climate; suitable for use as an annual for hanging pots outdoors under partial shade; and good weather tolerance with preferred temperature range of 15-25° C.

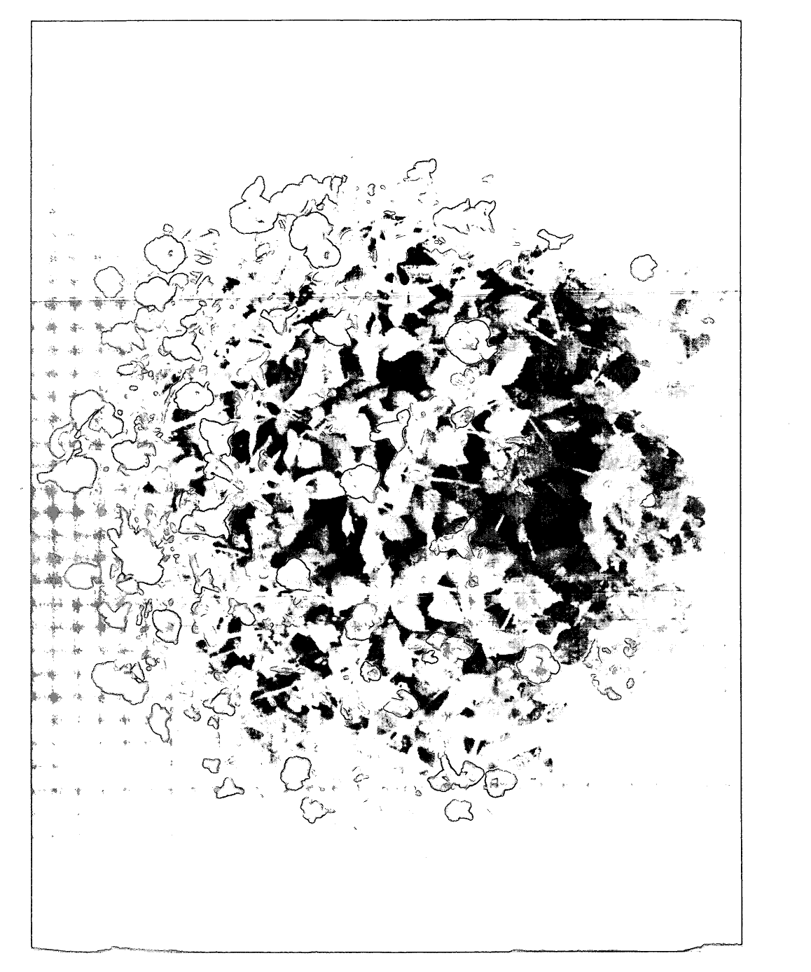

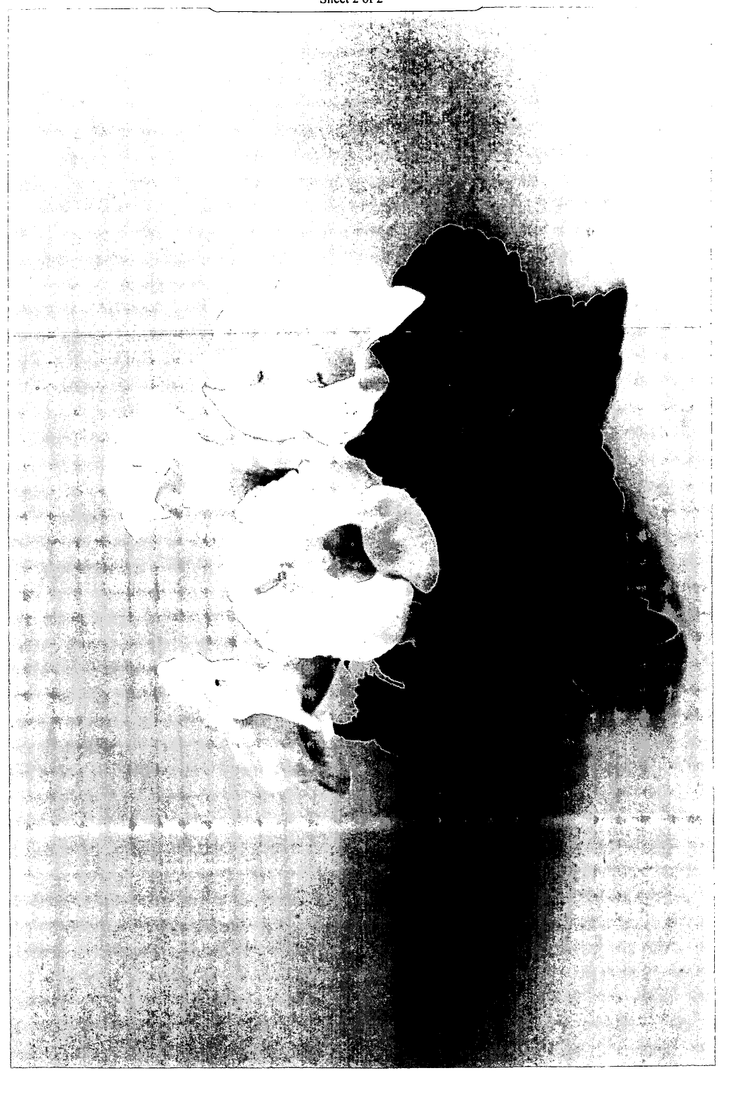

Genus and species of the plant claimed:

Torenia hybrida

Variety denomination:

Dantorwhite

### BACKGROUND OF THE INVENTION

The present invention relates to a new and distinct cultivar of Torenia plant of the Scorphulriacea family, hereinafter referred to by the cultivar name ‘Dantorwhite’.

The new cultivar originated in a controlled breeding program in Moshav Mishmar Hashiva, Israel. ‘Dantorwhite’ was discovered and selected by the inventor, Gabriel Danziger, as a flowering plant in a controlled environment in Moshav Mishmar Hashiva, Israel.

‘Dantorwhite’ is an induced mutant of the female cultivar Torenia ‘Rose Moon’ (unpatented), selected in a cultivated environment in Moshav Mishmar Hashiva, Israel. Mutation arose by radiation, as a side shoot, stabilized by removing cutting from the mutated sector of the treated plant.

Asexual reproduction of the new cultivar by soft tip cuttings was first performed in September, 2002 in Moshav Mishmar Hashiva, Israel and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be basic characteristics of ‘Dantorwhite’ which in combination distinguish this Torenia as a new and distinct cultivar:

1. Shiny green foliage, dotted with plenty of delicate, cup shaped flowers in shades of white with a yellow throat;

2. Bush cascading, vigorous, compact rounded growth, densely branched;

3. Early flowering, flowers all year round in moderate climate;

4. Suitable for use as an annual for hanging pots outdoors under partial shade;and 5. Good weather tolerance with preferred temperature range of 15-25° C.

Of the many commercial cultivars known to the present inventor, the most similar in comparison to ‘Dantorwhite’ is the parental cultivar Torenia ‘Rose Moon’. Table 1 shows a comparison between ‘Dantorwhite’and ‘Rose Moon’.

TABLE 1

| Characteristic | ‘Dantorwhite’ | Torenia ‘Rose Moon’ |
|---|---|---|
| Flower color | White and Yellow | Light Red-Purple |
| Flower size (inch) | 1.2 × 1.4 | 1.2 × 1.4 |
| (width by length) | | |
| Growth habit | Bush cascading | Bush cascading |
| Flowering time | 8 weeks from planting | 8 weeks from planting |

‘Dantorwhite’ has not been observed under all possible environmental conditions. The phenotype of the new cultivar may vary significantly with variations in environment such as temperature, light intensity, and day length without any change in the genotype of the plant. The following observations, measurements and values describe the new cultivar as grown in Moshav Mishmar Hashiva, Israel under conditions, which closely approximate those generally used in commercial practice

### BRIEF DESCRIPTION OF THE DRAWING

The accompanying color photographs show typical flower and foliage characteristics of ‘Dantorwhite’, with the colors being as true as possible with illustrations of this type. The accompanying photographic illustrations show: The first drawing shows: a flowering plant of ‘Dantorwhite’ in its entirety. The second drawing shows a close up of ‘Dantorwhite’.

### DETAILED BOTANICAL DESCRIPTION

The following observations, measurements and values describe the new cultivar as grown in Moshav Mishmar Hashiva, Israel under conditions, which closely approximate those generally used in commercial practice. For use as a pot plant, optimal growth conditions include grown outdoors in moderate climate, and indoors during winter. Irrigation and fertilization on a regular basis. Growth in full sunlight and partial shade is required in mid summer. A fully grown plant takes 3 months, and blooms all year-round in moderate climate.

Color references are made to The Royal Horticultural Society Color Chart (RHS), except where general colors of ordinary significance are used. Color values were taken under daylight conditions at approximately noon in Moshav Mishmar Hashiva, Israel.
Propagation: Tissue culture and soft tip cuttings.
Plant:
   Form.—Bush cascading.
   Growth habit.—Rounded and compact.
   Height.—6 inches.
   Spreading area.—20 inches across.
   Blooming period.—All year round in moderate climate.
   Cold tolerance.—Frost tender, plants may be damaged by temp. below 5° C.
   Vigor.—Fully grown plant takes 3 months during summer.
Stem:
   Color.—Yellow-Green 144-A.
   Diameter.—0.1 inch.
   Pubescence.—Few.
   Length of internodes.—1.5 inches.
Foliage:
   Overall shape.—Deltoid Base: Cordate Tip: Acuminate Margin: Serrate.
   Color.—Center color of leaf: Yellow-Green 147-A Upper side: Yellow-Green 147-A Under side: Yellow-Green 147-B
   Size of leaf.—Width: 1.0 inch Length: 1.5 inches Petioles 0.3 inch, Yellow-Green 144-A Leaf Texture: Convex Venation: None
Flowers:
   Blooming period.—All year round in moderate climate.
   Flowering stem.—Flowers appear along the growing stems.
   Orientation at opening.—Slantly upward.
   Type.—Single.
   Shape.—Funnel form.
   Petals.—Size: 1.2 inches (width) X 1.4 inches (length) Shape: Corolla tube cylindrical or often broadened above, 2-lipped, the upper lip faintly 2-lobed, the lower lip 3-lobed. Color: The corolla is white with a yellow blotch at the lower lip and a yellow throat. Upper side: RHS 155 C, RHS 1 A Under side: RHS 155 C, RHS 3 C Margin: RHS 155 C Throat: RHS 1 A
   Peduncle.—Length: 0.3 inch Color: Yellow-Green 144-A
   Bud.—Shape: Oblong cylindrical ellipsoid 5 winged Size: Length: 0.8 inch Diameter: 0.3 inch Color: RHS 4 D
Reproductive organs:
   Stamen.—2 pairs, white RHS 155 C.
   Style.—White RHS 155 C.
   Anther.—White RHS 155 C.
   Filament.—White RHS 155 C.
   Ovaries.—1, green RHS 143-B.
Seed/fruit: None.
Pest/disease resistance and/or susceptibility: A standard preventative spray program is sufficient as no susceptibility was observed.

I claim:

1. A new and distinct Torenia plant named ‘Dantorwhite’ as illustrated and described herein.
